(19) United States
(12) Patent Application Publication
(10) Pub. No.: US 2025/0058121 A1
(43) Pub. Date: Feb. 20, 2025

(54) SYSTEMS AND METHODS FOR TARGETING AND DOSING SPINAL CORD STIMULATION

(71) Applicant: Boston Scientific Neuromodulation Corporation, Valencia, CA (US)

(72) Inventors: Tianhe Zhang, Studio City, CA (US); Rosana Esteller, Santa Clarita, CA (US); Que T. Doan, West Hills, CA (US)

(73) Assignee: Boston Scientific Neuromodulation Corporation, Valencia, CA (US)

(21) Appl. No.: 18/804,639

(22) Filed: Aug. 14, 2024

Related U.S. Application Data
(60) Provisional application No. 63/532,809, filed on Aug. 15, 2023.

(51) Int. Cl. A61N 1/36; A61N 1/05

(52) U.S. Cl. CPC A61N 1/36139; A61N 1/0551; A61N 1/36071; A61N 1/3615; A61N 1/36171; A61N 1/36175; A61N 1/36178; A61N 1/36185; A61N 1/36132

(57) ABSTRACT

Methods and systems for performing spinal cord stimulation in the cervical spine while sensing for an evoked response, such as an evoked synaptic action potential (ESAP) at a different location from the location of stimulation. A first lead may be positioned in the cervical spine for issuing therapy pulses, and sensing is performed at a thoracic or lumbar location, or a caudal cervical location relative to the location of stimulation. First and second leads may be used in a single system, or two separate systems, one for stimulation and another for sensing, may be used.

### CROSS REFERENCE TO RELATED APPLICATIONS

The present application claims the benefit of U.S. Provisional Patent Application No. 63/532,809, filed Aug. 15, 2023, titled SYSTEMS AND METHODS FOR TARGETING AND DOSING SPINAL CORD STIMULATION, the disclosure of which is incorporated herein by reference.

### BACKGROUND

Implantable and/or wearable stimulations systems for the treatment of various diseases and disorders of the neurological system have proven effective in a wide variety of ways. For example, spinal cord stimulation (SCS) systems are accepted treatments for chronic pain syndromes. An SCS system typically includes an Implantable Pulse Generator (IPG). The IPG is coupled to tissue-stimulating electrodes carried on the distal end of one or more electrode leads that are implanted near the spinal column. The proximal ends of the one or more leads are tunneled through the patient's tissue to a location such as the buttocks where the IPG is implanted. The proximal ends of the leads are coupled to the IPG to provide electrical stimulation from the electrodes to alleviate a patient's symptoms, such as chronic back pain.

Historically, SCS has been delivered with a lead typically placed at the level of the thoracic vertebrae. Stimulation has historically been provided without the use of electrical feedback of an evoked biological response. There is interest in new implant locations, and also in new and alternative methods of sensing evoked response of the patient's neural system.

### OVERVIEW

The present inventors have recognized, among other things, that a problem to be solved is the need for new and/or alternative placements for SCS leads and electrodes. One solution is to place the lead at a cervical position with the intent to alleviate thoracolumbar symptoms and/or diffuse or visceral symptoms. Monitoring of an evoked response of the patient is proposed as well, and may include monitoring of the evoked compound action potential (ECAP), the evoked synaptic action potential (ESAP), or any other evoked signal in a patient's neural system.

An illustrative and non-limiting example takes the form of a system for providing electrical stimulation to a patient's spinal cord to treat pain in the patient comprising: one or more leads implantable in the patient's spinal column, each lead having a proximal end, a distal end, and a plurality of electrodes near the distal end; and a pulse generator connectable to the proximal ends of the one or more leads, the pulse generator comprising a housing containing control circuitry configured to: delivering a first stimulation signal using a first one or more of the electrodes to the patient's spinal cord at a stimulation site; sensing for an evoked response signal with a second one or more of the electrodes at a sensing site caudal to the stimulation site.

Additionally or alternatively, the pulse generator is configured to deliver the first stimulation signal to a cervical spinal location, and to sense for an evoked response at a thoracic or lumbar spinal location.

Another illustrative and non-limiting example takes the form of a system for providing electrical stimulation to a patient's spinal cord to treat pain in the patient comprising: one or more leads implantable in the patient's spinal column, each lead having a proximal end, a distal end, and a plurality of electrodes near the distal end; and a pulse generator connectable to the proximal ends of the one or more leads, the pulse generator comprising a housing containing control circuitry configured to: delivering a first stimulation signal using a first one or more of the electrodes to the patient's spinal cord at a stimulation site; sensing for an evoked response signal with a second one or more of the electrodes at a sensing site rostral to the stimulation site.

Additionally or alternatively, the pulse generator is configured to deliver the first stimulation signal to a thoracic spinal location, and to sense for an evoked response at a cervical spinal location.

Another illustrative and non-limiting example takes the form of a system for providing electrical stimulation to a patient's spinal cord to treat pain in the patient comprising: one or more leads implantable in the patient's spinal column, each lead having a proximal end, a distal end, and a plurality of electrodes near the distal end; and a pulse generator connectable to the proximal ends of the one or more leads, the pulse generator comprising a housing containing control circuitry configured to: delivering a first stimulation signal using a first one or more of the electrodes to the patient's spinal cord at a stimulation site; sensing for an evoked response signal with a second one or more of the electrodes at a sensing site at least one vertebral level away from the stimulation site.

Additionally or alternatively, the control circuitry is configured to delay at least 2 milliseconds from the issuance of the first stimulation signal, before sensing for the evoked response. Additionally or alternatively, the one or more leads includes a first lead and a second lead, wherein the control circuitry is configured to issue the first stimulation signal using at least one electrode on the first lead, and to sense the evoked response signal using at least one electrode on the second lead. Additionally or alternatively, the control circuitry is configured to deliver a second stimulation signal after the first stimulation signal and in response to sensing the evoked response signal, wherein the control circuitry is configured to deliver the second stimulation signal with at least one change to a frequency, a pulse width, an amplitude, a repetition rate, or a duty cycle of the first stimulation signal.

Additionally or alternatively, wherein the control circuitry delivers the first stimulation signal at the first location using a first configuration of electrical currents or voltages output across the first one or more of the electrodes, and, if the evoked response signal is detected at a first sensing site, but not at a second sensing site, the control circuitry is configured deliver a second stimulation signal after the first stimulation signal using a second configuration of electrical currents or voltages that is different from the first pattern, and to sense for the evoked response to occur at the second sensing site. Additionally or alternatively, the control circuitry is configured to determine whether the evoked response occurs at a first sensing site and, if not, to adjust a stimulation parameter used when delivering the first stimulation signal. Additionally or alternatively, the control circuitry stores a mapping of evoked signal responses to stimulation, and, on detecting the evoked response signal, uses the mapping to determine whether the first stimulation signal caused a desired evoked response.

Additionally or alternatively, the control circuitry is configured to sense an evoked synaptic action potential (ESAP) as the evoked response. Additionally or alternatively, the control circuitry is configured to identify changes in the ESAP and, in response to an identified change in the ESAP, to modify one or more therapy settings before delivering a second stimulation signal after the first stimulation signal. Additionally or alternatively, the control circuitry is configured to: sense for and detect an evoked compound action potential within two milliseconds of delivery of the first stimulation signal; and sense for and detected an evoked synaptic action potential more than two milliseconds after delivery of the first stimulation signal.

Another illustrative and non-limiting example takes the form of a method for providing electrical stimulation to a patient's spinal cord to treat pain using an IPG system comprising one or more leads implanted in the patient's spinal column, each lead comprising a plurality of electrodes, the method comprising: implanting a first lead into the spine, the first lead having a plurality of electrodes thereon; implanting a second lead into the spine, the second lead having a plurality of electrodes thereon; issuing a first therapy pulse from the first lead; and after the first therapy pulse, using the second lead to detect a first evoked response.

Additionally or alternatively, the method further includes using the first lead to detect a second evoked response, the second evoked response being an evoked compound action potential. Additionally or alternatively, the method includes using the second evoked response to determine whether the first therapy pulse caused a neural response in the patient; and using the first evoked response to determine whether the first therapy pulse is causing the neural response at a desired location in the patient. Additionally or alternatively, the method includes determining a location of the first evoked response; determining the location of the first evoked response is not a desired location; and adjusting a selection of electrodes on the first lead used for issuing the first therapy pulse, and issuing a second therapy pulse with the adjusted selection of electrodes. Additionally or alternatively, the step of using the second lead to detect the first evoked response comprises waiting at least two milliseconds after the stimulus is delivered to initiate sensing for the first evoked response. Additionally or alternatively, the method includes determining a location of the first evoked response; and using the location of the first evoked response to identify a dermatome of the patient that is being affected by the first therapy pulse.

Another illustrative an non-limiting example takes the form of a method of configuring neural stimulation in a patient, the method comprising: issuing a plurality of electrical stimuli using a first lead implanted at a first location in the patient's spinal column, while varying a central point of stimulation; as the plurality of electrical stimuli are issued, sensing for evoked responses to the plurality of electrical stimuli using electrodes on a second lead implanted at a second location in the patient's spinal column; and storing a mapping of central point of stimulation and evoked responses thereto.

Additionally or alternatively, the step of issuing the plurality of electrical stimuli is performed with the patient sedated, such that the plurality of electrical stimuli can be issued at amplitude levels the patient would find uncomfortable. Additionally or alternatively, the step of sensing for evoked responses comprises waiting at least two milliseconds after a respective one of the plurality of electrical stimuli is delivered before sensing for a corresponding evoked response.

Additionally or alternatively, the evoked responses are evoked synaptic action potentials. Additionally or alternatively, the first lead is in the cervical spine, and the second lead is in the thoracic or lumbar spine. Additionally or alternatively, the second lead is caudal to the first lead. Additionally or alternatively, the electrical stimuli are issued, and the evoked responses are sensed, at least one vertebral level away from one another. Additionally or alternatively, the plurality of stimuli are generated by an external pulse generator that is coupled to the first lead.

Another illustrative, non-limiting example takes the form of a method of treating a patient with a neuromodulation system having at least one lead with a plurality of electrodes located in the spinal column of the patient, the method comprising: delivering a first stimulation signal using a first one or more of the electrodes to the patient's spinal cord at a stimulation site; sensing for an evoked response signal with a second one or more of the electrodes at a sensing site caudal to the stimulation site.

Additionally or alternatively, the stimulation site is at a cervical spinal location, and the sensing site is at a thoracic or lumbar spinal location.

Another illustrative and non-limiting example takes the form of a method of treating a patient with a neuromodulation system having at least one lead with a plurality of electrodes located in the spinal column of the patient, the method comprising: delivering a first stimulation signal using a first one or more of the electrodes to the patient's spinal cord at a stimulation site; sensing for an evoked response signal with a second one or more of the electrodes at a sensing site rostral to the stimulation site.

Additionally or alternatively, the stimulation site is at a thoracic spinal location, and the sensing site is at a cervical spinal location.

Another illustrative and non-limiting example takes the form of a method of treating a patient with a neuromodulation system having at least one lead with a plurality of electrodes located in the spinal column of the patient, the method comprising: delivering a first stimulation signal using a first one or more of the electrodes to the patient's spinal cord at a stimulation site; sensing for an evoked response signal with a second one or more of the electrodes at a sensing site at least one vertebral level away from the stimulation site.

Additionally or alternatively, the step of sensing for an evoked response signal comprises delaying at least 2 milliseconds from the issuance of the first stimulation signal, before sensing for the evoked response. Additionally or alternatively, the stimulation system comprises first and second leads, and the first one or more electrodes are on the first lead, and the second one or more electrodes are on the second lead.

Additionally or alternatively, the method includes delivering a second stimulation signal after the first stimulation signal and in response to sensing the evoked response signal, wherein the second stimulation signal is delivered with at least one change to a frequency, a pulse width, an amplitude, a repetition rate, or a duty cycle of the first stimulation signal. Additionally or alternatively, the first stimulation signal is issued with a first central point of stimulation, and the method further comprises: determining a location of the evoked response signal; and in response to the location of the evoked response signal not matching a desired location, issuing a second stimulation signal at a second central point of stimulation different from the first central point of stimulation.

Additionally or alternatively, the method includes determining whether the evoked response occurs at a first sensing site and, if not, to adjust a stimulation parameter used when delivering the first stimulation signal. Additionally or alternatively, the method includes making an adjustment to a therapy setting in response to sensing the evoked signal response by reference to a mapping of evoked signal responses to stimulation.

Additionally or alternatively, the evoked signal response is an evoked synaptic action potential (ESAP). Additionally or alternatively, the method includes analyzing the ESAP to identify a change in the ESAP and, in response to a change in the ESAP, modifying one or more therapy settings before delivering a second stimulation signal after the first stimulation signal.

Additionally or alternatively, the method includes sensing for and detecting an evoked compound action potential within two milliseconds of delivery of the first stimulation signal.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows various functional components of an illustrative neurostimulation system;

FIG. 2 shows an illustrative implantable stimulator and electrodes;

FIG. 3 shows an illustrative spinal cord stimulation system as implanted;

FIG. 4 shows a graph of multiple evoked potentials produced by SCS;

FIGS. 5-10 show stimulation and sensing examples; and

FIGS. 11, 12, 13a-13b and 14 illustrate methods using evoked signals to manage patient stimulation and therapy.

### DETAILED DESCRIPTION

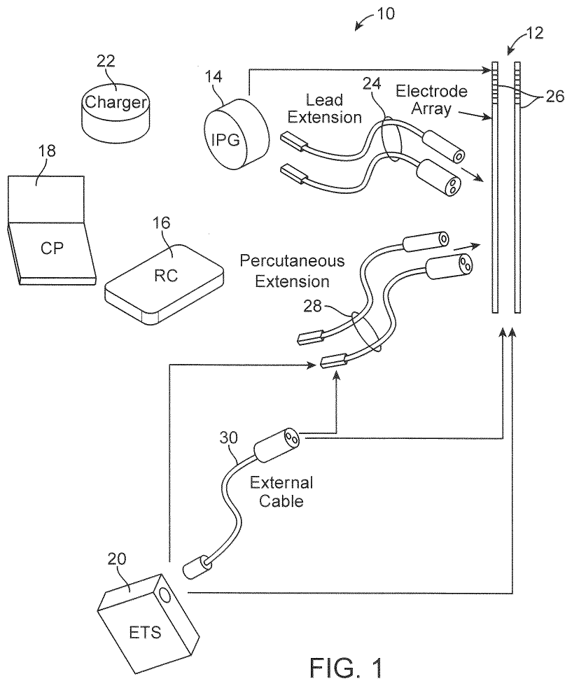

FIG. 1 shows a system for providing neurological therapy, which may be used, for example, as spinal cord stimulation (SCS), deep brain stimulation (DBS), peripheral nerve stimulation (PNS), or functional electrical stimulation (FES). The system 10 includes electrodes 12 configured for coupling to an implantable pulse generator (IPG) 14. The IPG may communicate with one or more of a patient remote control (RC) 16, a clinician programmer (CP) 18, and/or a charger 22. An external testing system (ETS) 20 may also be provided for testing therapy parameters prior to implantation of the IPG, using percutaneous extensions 28 and, as needed, an external cable 30 to couple to the implantable electrodes 12. If needed, lead extensions 24 may be used to couple the IPG to the implantable electrodes 12.

As shown in FIG. 1, the implantable electrodes may include arrays of electrode contacts on linear leads 26; in other examples, paddle leads may also be used. One, two or even four leads 12 may be provided, with up to 32 contacts on the leads 26, plus an additional contact in the form of the housing of IPG 14, available in modern systems. More or fewer contacts on more or fewer leads may be provided depending the particular system.

The IPG 14 can couple directly to the leads 26 or may be coupled via the lead extensions 24, depending on the positioning of each element as implanted. The IPG may include a rechargeable battery and charging coil to allow recharging when placed in proximity to the charger 22. Alternatively, the IPG may use a non-rechargeable battery and omit the charging coil and charger 22 from the system. In some examples, the IPG may be externally powered and omits a battery entirely.

The CP 18 can be used by a physician to manipulate the outputs of the IPG 14 and/or ETS 20. For example, the CP 18 can be used by the physician to define a therapy regimen or program for application to the patient. Multiple programs may be facilitated and stored by the IPG 14 or ETS 20; in some examples, the RC 16 may store the programs to be used. Communication amongst the IPG 14, RC 16, CP 18, ETS 20 and Charger 22 may use any suitable protocol such as wireless RF telemetry, inductive communication, Bluetooth, etc.

The RC 16 may be used by a patient to enable or disable therapy programs, to select between available programs, and/or to modify the programs that are available for use. For example, in some embodiments a patient may use the RC 16 to activate a stored program and then manipulate therapy by increasing or decreasing therapy strength and/or changing therapy location, within limits set by the physician.

The outputs of the IPG 14 may be manipulated in a closed loop. For example, a therapy regimen may be defined by a physician as described above. In addition to the therapy regimen, certain adjustments to stimulation parameters may be stored in the IPG 14, ETS 20, or RC 16. These programs may be facilitated in response to electric potentials as explained in more detail below.

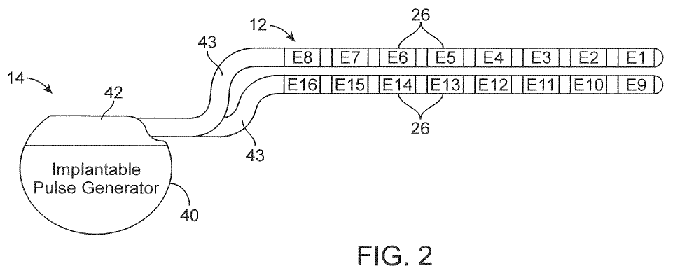

FIG. 2 shows an implantable stimulator and electrodes. As shown in the closer detail here, the IPG 14 may include a canister 40 and header 42. The canister 40 is conductive in most examples, using biocompatible materials such as titanium and/or stainless steel, for example, to allow use as an electrode when implanted. The header allows removable connection to the lead 12, which in this example may have a bifurcation or yoke allowing two segments 43 to extend therefrom, to two arrays 26 at the distal end of the lead 12. The electrode arrays 26 can be numbered as shown to facilitate ease of understanding when programming, with, for example, one array marked electrodes E1 to E8 on one of the lead segments 43, with E1 being distalmost. Other conventions may be used.

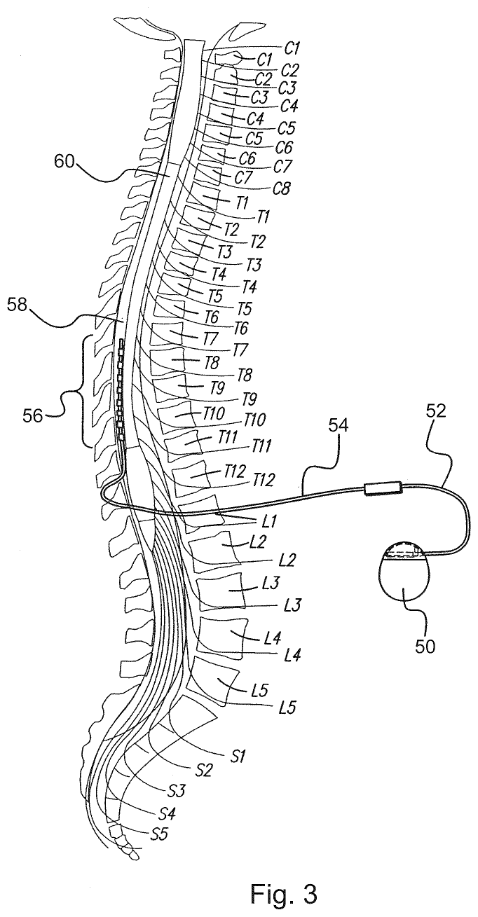

FIG. 3 shows an illustrative spinal cord stimulation system as implanted. In this example, an IPG 50 may be placed near the buttocks or in the abdomen of the patient, with or without a lead extension 52 for coupling to the lead(s) 54 that enter the spinal column. Region 56, at about the level of the lower thoracic or upper lumbar vertebrae may serve as an entry point to the spinal column, where the distal end of the lead 54 with an electrode array may be placed close to the spinal cord 58. Other locations for the IPG 50 and/or lead 54 may be used.

The standard approach to therapy in systems similar to those shown in FIGS. 1-3 has been that the IPG 14 (and ETS 20) may offer current controlled or voltage-controlled therapy comprising either biphasic square waves or monophasic square waves having passive recovery. In general, the amount of current out of an electrode should zero out over time to avoid encouraging corrosion at the electrode-tissue interface. For this reason, biphasic pulses, or monophasic pulses with a passive recovery period are typically used.

The leads 54, as shown in FIG. 3, are often placed in the thoracic region of the spinal column. Afferent signals, such as pain signals, transmit toward the brain along the spinal column. Some such signals do not enter the spinal column at or caudal to the thoracic lead position usually used. As a result, SCS delivered in the thoracic spine cannot affect pain signals that reach the spinal column at such higher positions.

Much of the therapy delivered in SCS is provided with no feedback or only indirect feedback. For example, during some testing and configuration procedures, a patient may consciously respond to questions regarding therapy benefits or side effects, interposing both the delay time needed for a patient to perceive stimulus effects, as well as adding uncertainty due to subjective factors to the feedback. Outside of the SCS (and other neural therapy) contexts, implantable stimulus systems are able to be used with direct means of monitoring evoked responses to therapy. For example, a pacemaker senses electrically whether a pace output has captured sufficient heart muscle tissue to evoke a contraction or heartbeat. Sensing of evoked responses in neural therapy is highly desired. New and improved ways of stimulation and sensing are desired.

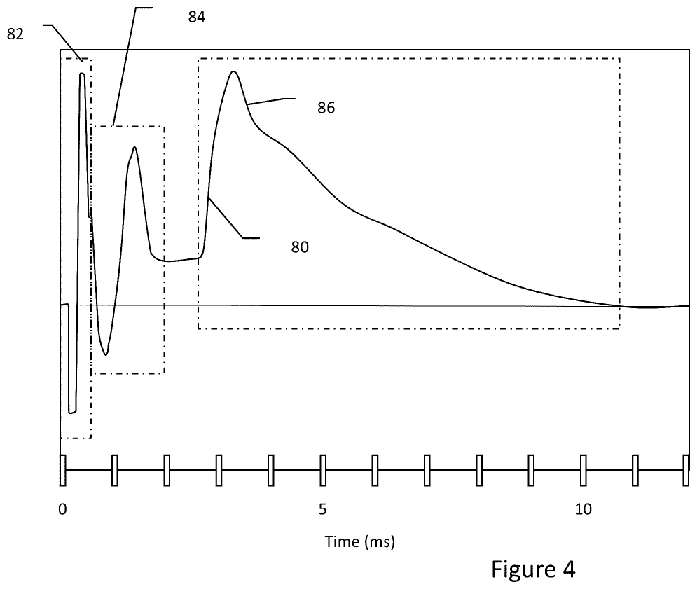

FIG. 4 illustrates a recording in graphic form of the signals that can be sensed in association with spinal cord stimulation, shown as the electro-spino-gram (ESG) 80. A stimulation artifact 82 occurs first in time, in the range of less than a millisecond after stimulus delivery. An evoked compound action potential (ECAP) is shown at 84, occurring about 0.5 to 2 milliseconds after the stimulus is delivered. The ECAP 84 occurs due to simultaneous firing of a population of nerve fibers in the region of stimulation. An evoked synaptic action potential (ESAP) 86 is also observed, and occurs in a time range of about 3 to about 10 milliseconds after therapy is output in this graphic.

Without being bound by theory, the inventors hypothesize that the ESAP originates from synapses and/or are evoked by synapses that connect dorsal column axons with neurons of the dorsal horn. Notice that the ESAP 86 occurs later than both the artifact 82 and the ECAP 84. Accordingly, the contribution of the ESAP to the ESG may be distinguished from the ECAP based on the time window during which the signal is recorded.

Another distinguishing feature of ESAPs is that they are generally most prominently observed with consistent, relatively unchanging amplitudes when the evoking stimulation frequency is ultra-low, for example, about 10 Hz or less. At higher frequencies, the amplitude of the ESAP is significantly reduced after only a small number of periods, though the ESAP may still appear sporadically. In some embodiments, the ESAP amplitude evoked with stimulation at 50 Hz starts decreasing after about the fourth pulse and then remains at smaller settled amplitude. With 10 Hz stimulation, the amplitude decreases more slowly, if at all. This is contrasted with ECAPs, which retain their magnitude and morphology even when SCS is applied at relatively higher frequencies (e.g., 50 Hz). Also, ESAPs are also correspondingly wider than the ECAPs. ECAP width (defined by N1 to P2 width) may only be 1-5 ms, whereas ESAP width, defined roughly as the width of the large positive phase, may be or exceed 5-10 ms.

Aspects of this disclosure relate to methods and systems for sensing, recording, characterizing, and using evoked signals to control therapy. The evoked signals may be, for example, and without limitation, ECAP or ESAP signals. For example, aspects of the disclosure involve using one or more features of the ECAP or ESAP as a feedback control variable for adjusting simulation parameters, as an indication of pain, and/or determining proper placement of stimulation. For example, stimulation parameters and/or stimulation location may be adjusted to maximize treatment efficiency and patient comfort based on one or more features of evoked signals. Alternatively, the parameters and/or stimulation location may be adjusted to minimize one or more features of the evoked signals, for example, if an ESAP feature is indicative of a side effect. In some embodiments, the evoked signal features may be used in conjunction with other sensed signals, such as other evoked signals (for example, using ESAP and ECAP together) and/or stimulation artifact signals. These sensed signals may be used for feedback control of the stimulation.

In one embodiment, the desired treatment location may be a particular dermatome/location on the body. That particular dermatome corresponds to a specific vertebral level. The system may sense the location of the ESAP and compare it to the intended vertebral level. If the ESAP location is different from the pre-determined vertebral level, the stimulation location may be changed.

In another embodiment, a therapy may be delivered using a set of electrodes with a central point of stimulation at a first location, such as the cervical spine. An evoked response may be sensed at a location that is at least one, and preferably two or more, spinal levels spaced away from to the first location. In some examples, the location for sensing is caudal to the location of stimulation, such as in the example of a cervical stimulation and a thoracic or lumbar sensing location. In other examples, the sensing location may be rostral to the stimulation location. In some examples, a different spinal region is used for sensing than that used for stimulation, such as by issuing therapy using electrodes at a cervical location, and sensing in a thoracic or lumbar location, such as in the thoracolumbar region. Stimulation and sensing may be performed using two separate leads.

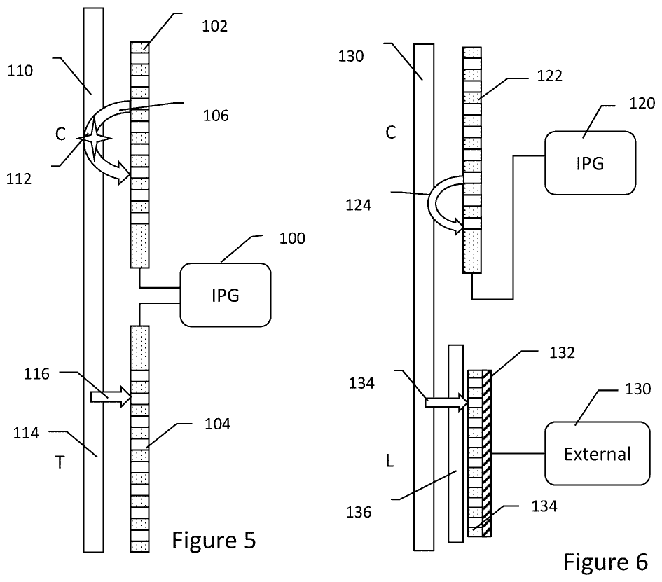

FIG. 5 illustrates an example of cervical spinal neurons being stimulated by an IPG system 100. A stimulation lead 102 is implanted in the cervical spine 110, and is connected to an IPG 100. A second lead 104 may be used by the IPG 100 for sensing in the thoracic spine 114. The IPG 100 issues a stimulus pulse, generally along delivered with a central point of stimulation (CPS) shown at 112, in the cervical spine. This may be achieved by using, for example, current steering such as disclosed in U.S. Pat. No. 6,909,917, the disclosure of which is incorporated herein by reference. The stimulation causes an evoked signal to be generated at the level where the dorsal column axons transmit to the dorsal horn, as shown at 116. In FIG. 5, the CPS 112 is positioned in a desirable location, and the amplitude of stimulation 106 (and/or pulse width or duty cycle) are sufficient to cause a neural response, eliciting an evoked response 116 in the thoracic region 114 of the spinal column. Lead 104 is used by the IPG to sense the evoked response 116 at a particular vertebral level.

As described above, various features of the evoked response may be used as feedback control for various stimulation parameters. For example, during a testing period, the evoked response signal may be monitored to determine, for the particular implant position and patient, the appropriate window after stimulus delivery for sensing to take place. In some examples, a blanking window may be used to focus only on a time window in which the evoked response occurs.

Blanking may vary with the evoked response to be monitored. For example, if an ECAP is to be sensed, blanking may include the stimulation delivery, and some short period thereafter, such as up to 0.5 ms, or 0.4 ms, or 0.3 ms, or 0.2 ms, or 0.1 ms, or blanking may optionally end with the termination of the stimulus delivery. For sensing an ESAP, on the other hand, a longer delay or blanking period may be used. For example, with an ESAP, sensing may be blanked for 1.0 to 4.0 ms after stimulation starts, or 0.5 to 2 ms after stimulation ends, for example. Frequency selective filtering may be used to remove the stimulation artifact, if desired, such as by the use of a digital filter.

Also, as noted, higher stimulation frequencies can affect or reduce the ESAP. If desired, monitoring for ESAP may be performed while stimulation is performed using therapy parameters that are in use at a given time. In other examples, ESAP sensing may be performed by reducing the stimulation frequency to facilitate the testing, as the purpose may be to determine whether the CPS 112 is located where it is intended to be, and modifying frequency would have little effect on the location of CPS 112.

Some examples may use relatively higher stimulation frequencies, such as in the kilohertz range. It may be noted, however, that stimulation at, for example, 1000 Hz implies a 1 ms period between stimuli, causing the stimulation signal to potentially interfere with sensing. For such a system, the use of frequency selective filtering to eliminate the stimulation artifact may be needed. Distance may also help to distinguish an evoked response from stimulation artifact, as the evoked response may be occur due to a signal being propagated by neural fiber action potentials, meaning its amplitude would not drop as a mere function of distance due to the attenuation of electrical fields of stimulation itself.

In some illustrative examples, testing for and observation of the ESAP, higher amplitudes of stimulus than those used for treatment, for example, of pain, may be used. For example, the ESAP may not be observable in some patients until an amplitude that is several times the activation threshold (at which an ECAP and/or a paresthesia may be observed), including as much as 10 to 15 times the ECAP threshold. For such uses, the testing and recording of ESAP signals may be performed in anesthetized patients. This need not always be the case, and it is theorized that the ESAP in human patients may instead be found at lower stimulus amplitudes. Some examples may include performing a calibration using high stimulus amplitudes to identify, for example, shape and/or timing characteristics of the ESAP, such as in a clinical or intraoperative setting, with subsequent monitoring at lower amplitudes, assuming that the ESAP may still be present with lower amplitude stimulus, just harder to identify due to reduced amplitude of the signal if, for example, reducing the stimulus amplitude reduces the population of neural cells that will generate the ESAP in response to stimulus.

Moreover, the evoked response (whether ECAP, ESAP, or other) may need to be extracted from other signals using, for example, machine learning algorithms, or using various analytical techniques such as principal component analysis, correlation waveform analysis, or wavelet transformations to parse out different portions of the signal corresponding to the desired/targeted evoked response and other signal components. Frequency selective filtering, for example, to remove higher frequencies, as well as a blanking interval, may be used. For example, the ESAP may be observed as a relatively wide signal, lasting for example, 3-10 milliseconds, with a starting point that is 4-10 milliseconds after stimulus delivery (the start delay may also depend on distance from stimulus site, as the ESAP may result from propagating ECAPs going up or down the spinal column, meaning that a greater distance form stimulation will require a greater delay as the ECAP must propagate to then induce the local ESAP). The wider signal of the ESAP may suggest a lower frequency content than other signal parameters, such that a low pass filter may be used to get rid of stimulation artifact, myopotentials, or other noise signals.

It may also be that the ESAP is observed at lower pulse repetition rates than are often used. For examples, relatively low frequency SCS can be performed, for example, with a pulse repetition rate of 40-60 pulses per second (PPS). ESAP signals, in some studies, have been observed when stimulus is delivered at, for example, 10 PPS, and cease to be readily observed as the frequency is increased to 60 PPS. Thus, special mode ESAP testing may be used by, for example, reducing repetition rate or frequency, while increasing stimulation amplitude, if desired. In an example, an SCS system may be capable of running multiple stimulation programs according to a schedule. A therapy program, operated at a physician selected frequency or repetition rate such as 60 Hz/PPS, may be scheduled to run repeatedly over a period of time. Every so often, however, an ESAP test program may be run, for example once a day, using a lower frequency or repetition rate such as 10 Hz/PPS, while keeping the pulse width and/or steering/CPS settings of the therapy program. Sensing and/or data recording may be used during the ESAP test program to determine presence or location of the ESAP, allowing therapy to be confirmed. Amplitude for the ESAP test program may be the same as, or different form (higher or lower) the amplitude used for the therapy program. Optionally, therapy may be paused prior to and/or after the ESAP test program runs, to mitigate or prevent carryover effects from and/or to the therapy program.

Sensing for the ECAP, on the other hand, may be more closely timed to the stimulation itself. ECAP sensing may be performed with or without modification of the underlying therapy, as desired, and may be performed on an ongoing basis as desired A determination may be made to select either ECAP or ESAP based on therapy parameters, for example, if therapy is being issued at a relatively higher frequency (over 100 Hz, for example), the ECAP may be preferred over the ESAP. This is just an example, and there is no particular requirement to use one or the other based on frequency in other examples. Other evoked potentials may call for different blanking duration, stimulation settings (such as amplitude, pulse width, rate, etc.) and/or duty cycle or therapy pause settings.

In an alternative example, the illustrative of FIG. 5 may be understood as upside-down, with the stimulation 106 being in a thoracic or lumbar location, and the sensing in a cervical location. That is, the sensing location may be rostral to the stimulation location. As another understanding, the sensing location may be spaced apart from the stimulation location by one or more vertebral levels.

FIG. 6 illustrates another example of cervical spinal neurons being stimulated by an IPG system 120. A stimulation lead 122 is implanted in the cervical spine 130, and is connected to an IPG 120. A sensing device 130 is coupled to a sensing patch 132 is located externally. An array of electrode contacts 134 is configured to transcutaneously sense from the lumbar spine through the skin 136. Transcutaneous sensing may be aided by the use of a hydrogel or other material to enhance tissue-electrode interface characteristics. The IPG 120 initiates a stimulation 124, and stimulates the cervical spine sufficiently and elicits an evoked response 138 in the lumbar spine. The evoked response is sensed through the skin 136 by the external device 130. As described above, various features of the evoked response may be used in a closed loop as feedback control for various stimulation parameters. Alternatively, a system as shown in FIG. 6 may be used to confirm desirable stimulation location, amplitude, etc. for the patient. In another example, stimulation may be modified, either to test new parameters or to return the system to efficacious stimulation if the system is not currently delivering desired benefits, for example. The IPG 120 and external device 130 may be configured for communication therebetween, if desired. The external device 130 may be dedicated to the sensing purpose, or may be any of a patent remote control, a clinician programmer, or a remote monitoring system used to monitor status of the IPG.

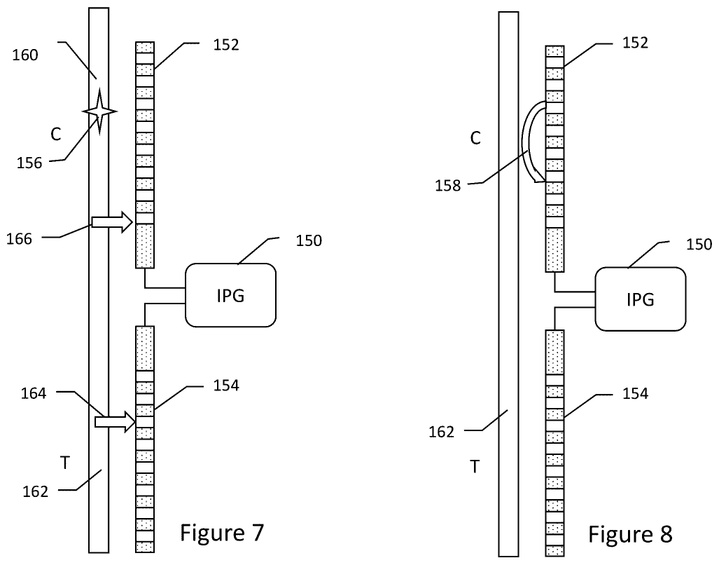

FIG. 7 illustrates an example of cervical spinal neurons being stimulated by an IPG system. A stimulation lead 152 is implanted in the cervical spine 160, and is connected to an IPG 150. The IPG 150 is also coupled to an implanted sensor lead 154, located in the thoracic spine 162. The IPG 150 initiates a stimulation through the stimulation lead 152. The resulting CPS is shown at 156, in/on/at the spinal cord. This causes an evoked response to propagate down the spinal cord, and results in an evoked response being generated and be sensed at location 164 on in the thoracic region 160. As described above, various features of the evoked response may be used as feedback control for various stimulation parameters.

It should be noted in the example of FIG. 7 that a separate lead may not be necessary for the sensing to take place. For example, the evoked response signal may be sensed at an inferior cervical location 166, using the same lead 152 as is used for issuing the stimulation pulse(s). In some examples, the evoked response is sensed caudal to the location of stimulation. In some examples, the evoked response is sensed at least a minimum distance away, such as at least one or at least two, or more, vertebral levels away from (preferably caudal, though not necessarily), the central point of stimulation. In another example, the sensing location is caudal to the most caudal electrode of the stimulation. In some examples, the sensing location at least one, two or more vertebral levels caudal of the most caudal electrode used for stimulation outputs. The two leads 152, 154 may be similar or identical in construction, or may be different, as desired. In some examples, electrodes on the sensing lead 154 may be spread out a greater distance than are commonly used in SCS for therapy delivery, so that multiple vertebrae can be traversed by the sensing electrode array, if desired.

In an example, the IPG 150 may include in its circuitry a switch array or multiplexor that would allow an of the electrodes on either lead 152, 154 to be used for sensing purposes, as well as switch arrays or multiplexors that would allow any of the electrodes to be used as a stimulation delivery electrode. Typically, different sets of electrodes would be used for stimulation than for sensing, though this is not an absolute necessity. IPG circuits usable for sensing and/or stimulation are well known to the skilled artisan, so details are not provided here. Some examples may be found in U.S. Pat. Nos. 6,181,969, 8,606,362, 8,620,436, and 9,259,574, and US Pat. App. Pubs. 2019/0175915, 2018/0071520, 2019/0083796, 2012/0095529, 2015/0157861, 2012/0092031, and 2012/0095519, the disclosures of which are incorporated herein by reference.

FIG. 8 illustrates an example of cervical spinal neurons not being sufficiently stimulated by an IPG system. The lead 152, does not stimulate the spine enough to elicit an evoked response in the thoracic spine 162, with the field produced as shown at 158 insufficient to place a CPS in/on the spine. The sensor lead 154 would not sense the evoked response following IPG stimulation and may use that information as feedback control for various stimulation parameters. For example, knowing the time at which an evoked response would be expected to occur following stimulation delivery, the IPG 150 would note that the expected signal was not present in the example shown in FIG. 8.

The cause of a failure to stimulate in FIG. 8 could include, for example, movement of the lead 152 creating additional distance between the target tissue and the lead, a change in neural response causing the needed current/voltage/power to increase, implementation of a requested change in therapy parameters, or other causes. A corrective action may be taken. For example, if stimulation is intended to be strong enough to elicit an evoked response, the IPG system may be configured to increase the current or voltage out of the stimulation lead electrode contacts to adjust treatment efficacy. In another example, pulse width may be modified by increasing or decreasing it, or duty cycle can be modified. In still another example, a loss of evoked response sensing may trigger further testing, such as by reducing the frequency of stimulation (temporarily) to allow retesting at a relatively low frequency, such as 10 Hz. Other tests, such as to determine the position of the lead electrically, may be triggered. A patent alert may be issued, and/or the patient may be requested to answer one or more questions regarding activities at the time and/or perceived changes in therapy efficacy.

“Loss” of the evoked response signal may be determined in several ways. In one example, absence of the evoked response at the expected time and location (where location is determined by the sensing electrodes in use) after a stimulation pulse can be identified as a loss of the evoked response. Some systems may sense for the evoked response by observing a peak signal during a defined time window, or by observing average amplitude during the defined time window. A drop in the average amplitude may be identified as loss of the evoked response. In some examples, evoked response may be observed on several consecutive pulsed outputs, and if absent for a predetermined period of time or quantity of continuous stimulus, loss of the evoked response may be identified. Other rules may be used.

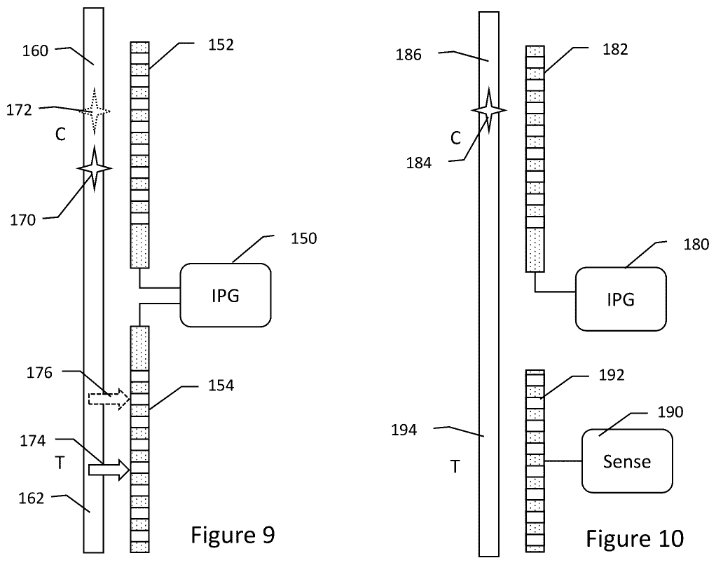

FIG. 9 illustrates an example of adjusting the stimulation field based on evoked response information. The stimulation lead 152 stimulates the cervical spine 160 and elicits an ESAP 174 in the thoracic spine 162. The sensor lead 154, senses the evoked response at a particular location. Here, the IPG 150 then recognizes that the vertebral location of ESAP does not correspond to the intended treatment dermatome. That is, in this example, the treated dermatome is considered to be the dermatome having nerve roots in the spinal column at the location of the evoked response 174 being sensed, as that is where the evoked response, which in this case may be an ECAP or an ESAP, is communicated from the spinal column to the dorsal horn. In some example, correlation of the evoked response site of sensing to particular dermatomes may be determined using mapping techniques in which stimulation is delivered, the location of the evoked response is determined, and patient feedback is obtained to determine which dermatome is being affected by the therapy.

Further in FIG. 9, a corrective action is performed. Here, the desired location for stimulation is shown at 176. In this example, using mapping or other data, the IPG determines that moving the CPS from 170 to 172 may also adjust the location that the evoked response would be sensed, and further that this is a desirable adjustment. The IPG can then adjust which electrodes are used to deliver therapy current/voltage pulses (as well as the voltage or current at each such electrode) in order to modify the CPS 172. The stimulation location is thus adjusted according to the evoked response signal location information. The spinal neurons are stimulated again, and the location of the evoked response signal changes because the location of the stimulation changes. The IPG 150 may repeat this process until the desired evoked response location is achieved. Such adjustment by the IPG may be autonomous, or may be performed with inputs from the patient and/or under physician control/guidance. This process may be performed while monitoring and adjusting the location of an evoked response such as the ECAP, the ESAP, or any other evoked neuronal response.

In some embodiments, the stimulation location may be adjusted by changing which electrode contacts on a lead are executing the stimulation program. In some embodiments, the stimulation location may be adjusted by changing which one or more of multiple implanted leads are executing the stimulation program. Some examples may also adjust stimulation location by changing the allocation of currents, or the designation of voltages, used on a plurality of electrodes, as desired.

FIG. 10 illustrates another example of a system configuration. The IPG 180 and stimulation lead 182 are connected and generate a stimulation output to produce a CPS as shown at 184. The stimulation lead 182 is shown implanted in the cervical spine 186. The sensor lead 192 is coupled to an implantable sensing module 190, and placed in the thoracic spine 194.

Here, the sensing module 190 may be an implanted module. In some examples, the sensing module 190 may lack a battery, and may be externally powered occasionally to allow system testing/verification of function to be performed, from time to time and as needed. The no-battery configuration may allow the sensing module 190 (which may have the electrodes placed thereon, rather than on a separate lead) to be miniaturized for implantation purposes. In other examples, the sensing module 190 may be a battery operated (rechargeable or non-rechargeable, optionally) device configured for sensing, or may be an IPG with similar functionality as IPG 180.

In FIG. 10, the spinal neurons 58 are stimulated, but not enough to cause the desired evoked response (ECAP, ESAP, or other) to be triggered or sensed. A corrective action may be taken, as described previously and below, such as by modifying the central point of stimulation, amplitude, pulse width, frequency, or other parameters. In some examples the sensing electrodes 192 are on a lead or paddle type device, or may instead be positioned on a housing for the sensing module 190. The sensing electrodes 192 may be positioned in the spine, on the spinal column, or in the vicinity of the spine. Preferably but not necessarily, the sensing electrodes 192 may extend a distance traversing at least one, or at least two, or at least three vertebrae.

In several examples above, the sensing electrodes are positioned inferior to, or caudal of, the stimulation electrodes. In another phrasing, the sensing electrodes may be positioned inferior to, or caudal of, the central point of stimulation, when a CPS is defined. In some examples, in addition to be caudal/inferior, the sensing electrodes are positioned one or two, or more, vertebrae away from the CPS, or one or two, or more, vertebral levels away from the most inferior (lead) electrode used to deliver therapy. For purposes of the description of sensing electrode relative positioning, or spacing from, stimulus electrodes, the housing electrode on the IPG may be ignored. The relative positioning may be reversed, if desired, so that the sensing electrodes are superior or rostral to the stimulation electrodes, using similar methods as discussed above.

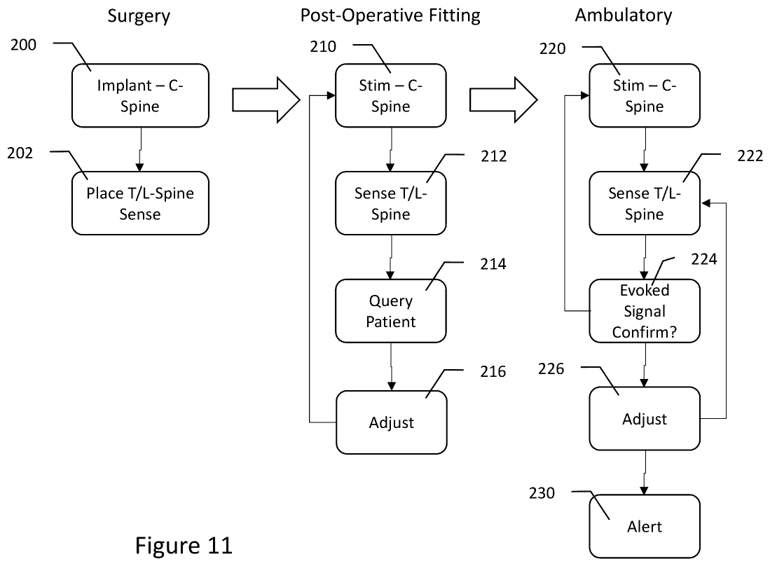

FIG. 11 illustrates in block form, an illustrative process of implanting, testing, and initiating treatment with an SCS device. Surgery is performed to implant a stimulation lead in the cervical spine, as indicated at 200. Block 200 may also include implanting a pulse generator in an appropriate position. While most SCS devices are implanted with the pulse generator in the buttocks, with the cervical spinal position, other locations may be more viable. The inframammary crease (under the arm) has been used to implant a pulse generator, as well as locations on the upper chest such as in the pectoral region. In such positions, the IPG may be placed on the right side of the patient, so as to preserve the left side of the upper torso for cardiac device implantation if ever needed (such as a pacemaker).

At 202, sensing electrodes are positioned in position allowing sensing of neural signals in the thoracic or lumbar spine. The sensing electrodes may be on or attached to a sensing module (FIG. 10) that is implanted. The sensing electrodes may instead be on a lead that is attached to the same pulse generator that was implanted in block 200, as shown in FIGS. 5 and 7-9. In other examples, the sensing electrode can be external, as shown above in FIG. 6.

To fit the stimulation post-operation, a series of test stimulations are performed to accurately map the patient's spine and neural response, so as to target the desired treatment location. In the fitting process, the cervical spine is stimulated as indicated at 210, and the subsequent ESG signals and information are sensed in the thoracic or lumbar spine, as indicated at 212. Sensing for evoked signals in the ESG may be performed by, for example, setting a sensing window with a start time in the range of 0.1 to 15 ms after stimulus is issued (start, midpoint, or end of the stimulation pulse) and a duration of about 3 ms to about 10 ms. In an illustrative example, an ESAP sensing window is set to begin at least 2 ms (range 2-4 ms) after the end of the stimulation signal and has a duration of 5 to 8 ms. Sensing for an ECAP signal may begin more or less immediately (such 0, 10, 25, 50, or 100 microseconds after the end of the therapy signal), as desired. Other evoked signal windows may be defined as needed.

The delay before starting sensing for the ESAP may be determined during testing and configuration of the patient's therapy. It may be expected that the ESAP sensed in a thoracolumbar position, or other evoked potential resulting from cervical stimulation, may be still later than 2 milliseconds, due to the time needed for either the ECAP, which can cause and ESAP, or the ESAP itself, to propagate from the cervical stimulation location to the location of sensing. Such a delay may also be affected by the potential for dispersion of the propagating neural signal; for example, an ECAP that lasts 1 millisecond at the location of stimulation may become extended for example to 2 milliseconds at a location several centimeters caudal in the spinal column.

The patient may be questioned as to if they felt any sensation, and where, as indicated at 214. The stimulation parameters being used are then adjusted, as indicated at 216. Adjustments may include changing pulse frequency or repetition rate, amplitude, pulse width, duty cycle, and/or electrode selection/steering (to affect CPS). Other variations can be made including switching between biphasic square waves with active recovery and monophasic square waves with passive recovery. Pulse shape can be modified, if non-square waves are available, and changes can be made to burst parameters, ramping up/down, or any other desired parameters of stimulation. Repeated stimulation, sensing and adjustments to various stimulation parameters may be made if necessary to target the desired treatment area and stimulation strength.

In some examples, the output signals used to test for ESAP signals may be larger than would be used for chronic therapy, such as 10 times or more greater amplitude than a signal found to induce ECAP signals, or 10 times or more greater amplitude than an amplitude found to induce paresthesia. Such high amplitude testing may be performed in an operational setting while the patient is subject to anesthesia and/or paralytics to prevent or limit muscle response to such higher amplitude signals. In other examples, lower amplitudes may be used; the ESAP may not be present in some patients when lower amplitudes are used, even if the ECAP or other evoked response signals can be identified.

After the surgery and post-operative fitting is complete, the system may continue to operate in the ambulatory setting. Here, as shown at 220, stimulation is delivered to the cervical spine. Sensing takes place again in the thoracic or lumbar spine as indicated at 222, using implanted or external sensing electrodes discussed above. If the evoked response is confirmed as being present and sensed at a location that is desired (such as that configured in the post-operative fitting), the method loops back to block 220. Sensing of the evoked response can be continuous, periodic (at intervals) or occasional (responsive to an event). For example, postural changes or certain activities may affect where the lead contacts are stimulating. When a postural change, or a change in activity level is sensed (such as by use of an accelerometer positioned in the IPG), evoked response sensing can be triggered to confirm continued desirable stimulation.

In some embodiments, the patient is able to adjust stimulation parameters with the patient RC. The patient may be able to execute programs set by a physician. When stimulation is turned on, or a setting is changed, or a new program is executed, evoked response sensing can be triggered. In some embodiments, the IPG system is configured to confirm presence and strength of evoked responses throughout treatment. If evoked response is outside of a set range, block 224 may trigger adjustments at block 226 until the sensed evoked response is back in the desired range. In some embodiments, the patient may wish to decrease stimulation strength while they sleep. The IPG system may be configured to adjust stimulation parameters at certain times of the day. Such adjustments may be performed using the evoked response testing regimen.

For example, if paresthesia is relied upon during waking hours by a patient, that may be a level of stimulation sufficient to trigger an evoked response that can be sensed at a separate location in the spine, such as with cervical stimulation and thoracic sensing, for example. Subthreshold therapy, delivered at a lower amplitude and without paresthesia while still providing therapeutic benefits, may be used for sleep or rest times for the patient. Alternatively, subthreshold therapy may be used all or most of the time. The evoked response signal monitor may be used to monitor evoked responses while reducing therapy amplitude, pulse width, duty cycle, repetition rate, or making other parameter adjustments to eliminate paresthesia. For example, amplitude can be reduced until the evoked response disappears from the sensed signals. Throughout a subthreshold or non-paresthesia therapy time period, the evoked response may be monitored to make sure that therapy remains sub-threshold. From time to time, amplitude may be increased until the evoked response is detected, after which therapy amplitude can again be reduced. When the evoked response is detected, the system may confirm that the evoked response is sensed at a desired or expected location and, if not, adjust the CPS as needed before or after reducing amplitude. In this way, the continued delivery of stimulation to the correct spatial location can be confirmed, without the patient interacting with the system consciously. It may be that some patients will not perceive paresthesia before the evoked response is sensed in such a procedure can confirm desired delivery of the subthreshold therapy without patient awareness. In some examples, rather than adjusting therapy amplitude in the preceding, pulse width or other parameter(s) may be adjusted.

In the event that therapy adjustments in block 226 are not effective to get the evoked response back to a desired range or location, or to get the evoked response to appear again if lost or not present, the system may issue an alert 230 to the patient and/or to a physician, using known methods for patient and physician alerts.

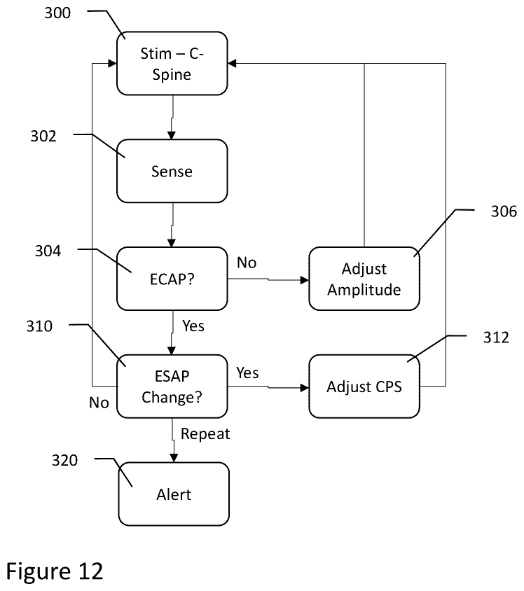

FIG. 12 shows another illustrative method. Here, both the ECAP and ESAP signals are analyzed. Stimulation is delivered in the cervical spine at 300. Signals are sensed, for example, at an inferior location of the cervical spine from where stimulation is delivered, or in the thoracic or lumbar regions. The method determines from the sensed signals whether the ECAP is present, at block 304. If there is no ECAP, then no stimulation is occurring, and so the amplitude of stimulation can be adjusted as indicated at 306 and the method returns to block 300.

If the ECAP is sensed at 304, the neural tissue is responding to the stimulation. The method then determines whether the ESAP has changed, relative to an initial ESAP signal, which may be from fitting or simply a previous iteration of the method. If no ESAP change, then stimulation is as desired and the method returns to 300 without adjustments. If the ESAP has changed at block 310, this may mean that while neural tissue is responding to stimulus, its not the correct or desired portion of neural tissue that is responding. The CPS can then be adjusted, as indicated at 312, and the method returns to block 300. If the ESAP does not return to the desired location/amplitude after several tries in block 312, the method may proceed to issue an alert, as indicated at 320, to the patient and/or patient's physician.

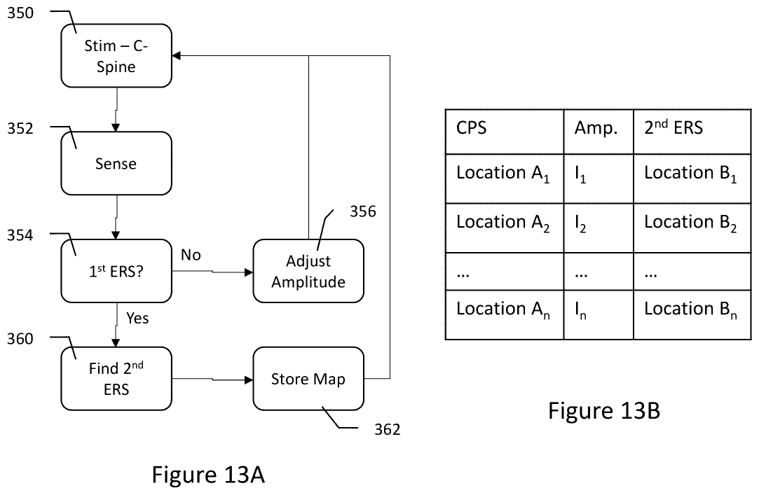

FIGS. 13A-13B illustrate a mapping method. Here, as shown at 350 in FIG. 13A, stimulation is issued in the cervical spine region, and sensing is performed at an inferior or more caudal position along the spine relative to the location of stimulation, as indicated at 352. The more caudal position, as discussed above, may be a lumbar, thoracic, or even lower cervical position. The method then determines whether a first evoked response signal (ERS) has been sensed, as indicated at 354. If there is no first ERS, the tissue is not responding, and so amplitude is adjusted as indicated at 356. If the first ERS is observed, then the location of a second ERS is determined, as indicated at 360.

For example, sensing can be performed across multiple sense channels at once, or maybe repeatedly performed along with repeated stimulus until the second ERS signal can be found. The second ERS signal may occur at spatially distinct locations depending on where the CPS is located. Then, in a trolling fashion, the CPS can be moved along the cervical spine, including in lateral directions, if a paddle electrode or two linear electrodes are placed, such that the CPS can be moved laterally, and/or up and down along the spinal column. A map can then be stored of which locations the second ERS is observed as the CPS is moved, along with amplitude settings sufficient to trigger the first ERS.

FIG. 13B shows a mapping of CPS, Amplitude, and ERS. If desired, a fourth column can be provided in which the specific dermatome of the patient, as indicated by querying the patient, associated with each row is identified. This type of mapping can be used to establish not only an overall neural mapping of patient responses, but also to determine how corrective actions can be taken when the CPS needs to adjusted in any of the above examples. That is, if the ERS is located at location B2, but is desired at location B1 (using the rows in FIG. 13B), the mapping can be used to determine which direction to move the CPS. The mapping may also aid in making amplitude adjustments to avoid under or over stimulating the patient as the CPS is moved.

In some examples, the first ERS of FIGS. 13A-13B is sensed proximate the stimulus delivery electrodes, such as being on the same lead, or adjacent to the stimulus delivery electrodes, and the second ERS is sensed at greater distance from the stimulus delivery electrodes, such as on a different lead, or at least one vertebral level away from the stimulus delivery electrodes. In another example, the first ERS is superior to or rostral to the stimulus delivery electrodes, and the second ERS is inferior to or caudal of the stimulus delivery electrodes. In still another example, the first ERS is both proximate to and rostral of the stimulus delivery electrodes, and is used to confirm that the therapy or stimulus delivery is causing some degree of evoked potential, and the second ERS is used to develop a mapping allowing a distant or remote sensing electrode to be set. More generally, the first, second, and/or other ERS may be related to or dependent on one another and, if the inter-relationship is known (for example, the ESAP seems to be linked to or dependent on the ECAP), the failure to sense one while sensing another may be indicative of a need to adjust or titrate therapy parameters, check sensing fidelity, and or to guide spatial steering, or other therapy modification, as described in several examples above.

The reasoning may be, optionally, to use the second ERS as a way of tracking therapy effect on a desired dermatome. The first ERS, being adjacent the stimulus, may be able to determine that an evoked response is occurring, but may not provide an indication of whether the therapy is directed to the desired dermatome. In some examples, each ERS is the same type of signal, just sensed at different locations (for example, both ECAP). In another example, the first ERS is an ECAP, and the second ERS is an ESAP or other evoked signal.

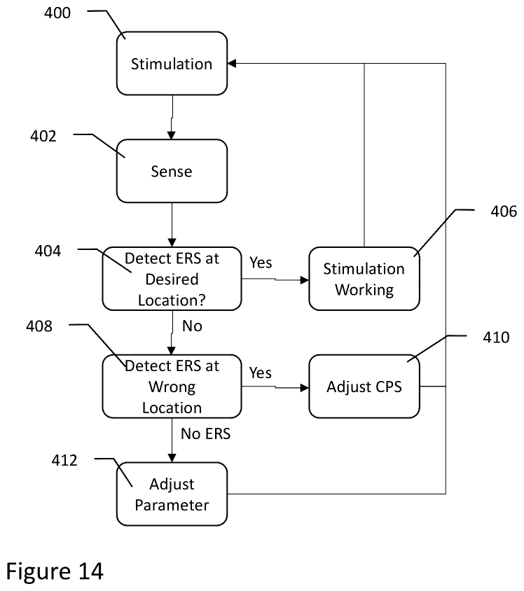

FIG. 14 shows another illustrative method. Here, stimulation is delivered at 400 using a set of stimulation parameters and steering settings. Sensing is performed at 402, to identify an evoked response signal (ERS). If the ERS is detected at a desired location, as queried at 404, the stimulation is working as designed/configured, as indicated at 406, and the method loops back to block 400. If the ERS is detected, but at the “wrong” location, as indicated at 408, then the central point of stimulation (CPS) can be adjusted as indicated at 410, and a second stimulation is attempted at 400 using the adjusted CPS.

If no ERS is detected, then other parameters, such as pulse width, repetition rate, amplitude, duty cycle, etc. are then adjusted, as indicated at 412, and stimulation is retried using the adjusted parameters, returning to block 400. For example, if neither of 404 or 408 detects an ERS, it may be that the stimulation amplitude is insufficient, and block 412 may include increasing stimulation amplitude or, alternatively, decreasing stimulation amplitude as it may be in some examples that the ERS is occurring at a location away from the sensing electrodes. In another example, if neither of 404 or 408 detects an ERS it may be that the pulse width is not providing desired degree of stimulation, and so block 412 may include increasing (or, alternatively, decreasing) the pulse width. Some examples may include reducing pulse repetition rate at block 412 if, for example, the ESAP is being monitored, as the ESAP may be of lesser amplitude or may not be sensed if the stimulation frequency is, for example, at 50 Hz or above; thus, block 412 may include reducing, at least temporarily, the pulse repetition rate or frequency to, for example, in the range of 5 to 20 Hz, or about 10 Hz.

In some examples, pulse shape may be modified instead of any of pulse width, amplitude, duty cycle, repetition rate and/or frequency, such as issuing square waves, ramped square waves, triangular outputs, semi-sinusoidal, or sinusoidal outputs, and changing from one shape to another, or changing a feature of a shape, such as increasing or decreasing a slope. In other examples, step 412 may include checking whether current stimulation parameters are different from those previously programmed by a physician. For example, when reaching block 412, the implantable device may determine that the patient has used the RC to modify therapy settings relative to those that were set by a clinician; if so, the settings may be reverted to those originally set by a physician. In other examples, each of CPS and other parameters may be modified in block 412.

In some examples, burst stimulation is issued. A “burst” is a set of therapy pulses delivered as a group using a pulse repetition rate, with quiescent periods between output groups. For example, fifteen pulses may be issued at 100 Hz (10 ms period from pulse start to pulse start), with a period of one second from the start of one group to the next; the intraburst and interburst frequencies/repetition rates may range widely. Modifications to a burst (at, for example, block 412) may include changes to pulse amplitude, shape or width, as desired, or may affect the number of pulses delivered in each burst grouping, or may affect the repetition rate within bursts or between bursts overall. With a burst therapy, for example, the priority of sensing one or more ERS signals may be adjusted to accommodate limitations on sensing. For example, while a burst therapy of 100 Hz or greater signal content may not be amenable to ESAP sensing, the ECAP may still be sensed, and so during a therapy, sensing to check the therapy may focus on only the evoked response that is likely to be available for sensing.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method for providing electrical stimulation to a patient's spinal cord to treat pain using an IPG system comprising one or more leads implanted in the patient's spinal column, each lead comprising a plurality of electrodes, the method comprising:
implanting a first lead into the spine, the first lead having a plurality of electrodes thereon;
implanting a second lead into the spine, the second lead having a plurality of electrodes thereon;
issuing a first therapy pulse from the first lead; and
after the first therapy pulse, using the second lead to detect a first evoked response.

2. The method of claim 1, further comprising using the first lead to detect a second evoked response, the second evoked response being an evoked compound action potential.

3. The method of claim 1, further comprising:
using the second evoked response to determine whether the first therapy pulse caused a neural response in the patient; and
using the first evoked response to determine whether the first therapy pulse is causing the neural response at a desired location in the patient.

4. The method of claim 1, further comprising:
determining a location of the first evoked response;
determining the location of the first evoked response is not a desired location; and
adjusting a selection of electrodes on the first lead used for issuing the first therapy pulse, and issuing a second therapy pulse with the adjusted selection of electrodes.

5. The method of claim 1, wherein the step of using the second lead to detect the first evoked response comprises waiting at least two milliseconds after the stimulus is delivered to initiate sensing for the first evoked response.

6. The method of claim 1 further comprising:
determining a location of the first evoked response; and
using the location of the first evoked response to identify a dermatome of the patient that is being affected by the first therapy pulse.

7. A method of configuring neural stimulation in a patient, the method comprising:
issuing a plurality of electrical stimuli using a first lead implanted at a first location in the patient's spinal column, while varying a central point of stimulation;
as the plurality of electrical stimuli are issued, sensing for evoked responses to the plurality of electrical stimuli using electrodes on a second lead implanted at a second location in the patient's spinal column; and
storing a mapping of central point of stimulation and evoked responses thereto.

8. The method of claim 7, wherein the step of issuing the plurality of electrical stimuli is performed with the patient sedated, such that the plurality of electrical stimuli can be issued at amplitude levels the patient would find uncomfortable.

9. The method of claim 7, wherein the step of sensing for evoked responses comprises waiting at least two milliseconds after a respective one of the plurality of electrical stimuli is delivered before sensing for a corresponding evoked response.

10. The method of claim 7, wherein the evoked responses are evoked synaptic action potentials.

11. The method of claim 7, wherein the first lead is in the cervical spine, and the second lead is in the thoracic or lumbar spine.

12. The method of claim 7, wherein the second lead is caudal to the first lead.

13. The method of claim 7, wherein electrical stimuli are issued, and the evoked responses are sensed, at least one vertebral level away from one another.

14. A method of treating a patient with a neuromodulation system having at least one lead with a plurality of electrodes located in the spinal column of the patient, the method comprising:
delivering a first stimulation signal using a first one or more of the electrodes to the patient's spinal cord at a stimulation site;
sensing for an evoked response signal with a second one or more of the electrodes at a sensing site caudal to the stimulation site.

15. The method of claim 14, wherein the stimulation site is at a cervical spinal location, and the sensing site is at a thoracic or lumbar spinal location.

16. The method of claim 14, wherein the step of sensing for an evoked response signal comprises delaying at least 2 milliseconds from the issuance of the first stimulation signal, before sensing for the evoked response.

17. The method of claim 14, wherein the stimulation system comprises first and second leads, and the first one or more electrodes are on the first lead, and the second one or more electrodes are on the second lead.

18. The method of claim 14, further comprising delivering a second stimulation signal after the first stimulation signal and in response to sensing the evoked response signal, wherein the second stimulation signal is delivered with at least one change to a frequency, a pulse width, an amplitude, a repetition rate, or a duty cycle of the first stimulation signal.

19. The method of claim 14, wherein the first stimulation signal is issued with a first central point of stimulation, and the method further comprises:
determining a location of the evoked response signal; and
in response to the location of the evoked response signal not matching a desired location, issuing a second stimulation signal at a second central point of stimulation different from the first central point of stimulation.

20. The method of claim 14, further comprising determining whether the evoked response occurs at a first sensing site and, if not, to adjust a stimulation parameter used when delivering the first stimulation signal.
